(19) United States
(12) Patent Application Publication
(10) Pub. No.: US 2026/0335452 A1
(43) Pub. Date: Oct. 29, 2026

(54) GLYCAN COMPLEX, DRUG CARRIER, AND DRUG DELIVERY SYSTEM

(71) Applicant: NATIONAL UNIVERSITY CORPORATION TOKAI NATIONAL HIGHER EDUCATION AND RESEARCH SYSTEM, Nagoya-shi, Aichi-ken (JP)

(72) Inventors: Satoshi NOZAWA, Nagoya-shi (JP); Naoya IWATA, Nagoya-shi (JP); Fumiko KAWASAKI, Tokyo (JP)

(73) Assignee: NATIONAL UNIVERSITY CORPORATION TOKAI NATIONAL HIGHER EDUCATION AND RESEARCH SYSTEM, Nagoya-shi, Aichi-ken (JP)

(21) Appl. No.: 19/114,332

(22) PCT Filed: Oct. 3, 2023
(86) PCT No.: PCT/JP2023/035984

§ 371 Date: Mar. 21, 2025

(30) Foreign Application Priority Data
Oct. 14, 2022 (JP) 2022-165577

(51) Int. Cl. A61K 47/61; A61K 47/54; A61K 47/62

(52) U.S. Cl. CPC A61K 47/61; A61K 47/545; A61K 47/62

(57) ABSTRACT

The glycan complex has a sugar chain to which a hydroxyapatite-binding molecule is bonded. The hydroxyapatite-binding molecule is at least one of an acidic peptide and estradiol.

### CROSS-REFERENCE TO RELATED APPLICATIONS

This application is national stage application of International Application No. PCT/JP 2023/035984, filed on Oct. 3, 2023, which designates the United States, and which claims the benefit of priority from Japanese Patent Application No. 2022-165577, filed on Oct. 14, 2022, the entire contents of which is incorporated herein by reference.

### TECHNICAL FIELD

The present invention relates to a glycan complex, a drug carrier, and a drug delivery system.

### BACKGROUND ART

Patent Literature 1 describes an ink that contains (1) alginic acid and/or a salt thereof, (2) water, and (3) at least one compound selected from a group consisting of a compound having a peptide bond and glycosaminoglycan. When the ink undergoes an inkjet process, gelation occurs and microbeads are formed. The application of the ink obtains satisfactory gel formation and satisfactory ink ejection.

In addition, Patent Literature 1 describes that the compound having a peptide bond and glycosaminoglycan have an excellent cell adhesion property and thus are expected to be used as a scaffold material in the regenerative medical field. Patent Literature 1 describes that the microbeads are used for development of a drug delivery system.

### CITATION LIST

### Patent Literature

Patent Literature 1: Japanese Laid-Open Patent Publication No. 2013-111401

### SUMMARY OF INVENTION

### Technical Problem

Gel microbeads have lower fluidity than liquid ink. Hence, when a drug is added to the microbeads so that the microbeads are used as a drug carrier, the drug is likely to stay in a predetermined location of the body. This allows the concentration of the drug to be increased locally.

The microbeads described in Patent Literature 1 locally increase the concentration of the drug due to the low fluidity of the gel. Therefore, as time elapses, decreases in the concentration cannot be avoided sufficiently. In the drug delivery system, there is room for improvement in locally increasing the concentration of a drug in a preferred manner.

Moreover, in Patent Literature 1, there is no consideration of a bone as the location of the body where the concentration of the drug is locally increased. When performing bone-related treatment such as for a fracture or osteoporosis, the effect of the treatment may be enhanced, and the drug dosage may be reduced by locally increasing the concentration of the drug around the bone.

A typical glycan tends to readily bind to a drug such as a growth factor. On the assumption that the glycan is used as a drug carrier in a drug delivery system, the concentration of the glycan around the bone needs to be increased in a preferred manner.

### Solution to Problem

In a first aspect, a glycan complex includes a glycan that binds to a hydroxyapatite-binding molecule. The hydroxyapatite-binding molecule includes at least one of acidic peptide and estradiol.

In a second aspect, in the glycan complex according to the first aspect, the acidic peptide includes at least one selected from aspartic acid and glutamic acid.

In a third aspect, in the glycan complex according to the first or second aspect, the glycan includes at least one selected from heparin, chondroitin sulfate, and hyaluronic acid.

In a fourth aspect, in the glycan complex according to the third aspect, the heparin is a low molecular weight heparin.

In a fifth aspect, in the glycan complex according to any one of the first to fourth aspects, the glycan further binds to at least one selected from a bone forming factor, a fibroblast growth factor, a vascular endothelial growth factor, and a hepatocyte growth factor.

In a sixth aspect, a drug carrier includes the glycan complex according to any one of the first to fifth aspects.

In a seventh aspect, a drug delivery system uses the glycan complex according to any one of the first to fifth aspects to increase a concentration of a drug around a bone.

### Advantageous Effects of Invention

The glycan complex according to the present invention increases the concentration of glycan around a bone in a preferred manner.

### BRIEF DESCRIPTION OF DRAWINGS

FIG. 1 is a schematic diagram of glycan complexes.

FIG. 2 is a reaction formula in which a residue of aspartic acid, corresponding to a hydroxyapatite-binding molecule, binds to a low molecular weight heparin, corresponding to a glycan.

FIG. 3 includes an upper part showing a micrograph of a rat in the upper part and a lower part showing a fluorescence image of the rat taken at the same position.

FIG. 4 includes micrographs for evaluating bone fusion when an artificial bone containing a glycan complex is disposed in the vicinity of the L4 and L5 vertebrae of the rat.

### DESCRIPTION OF EMBODIMENTS

An embodiment of a glycan complex according to the present invention will now be described.

The glycan complex includes a glycan that binds to a hydroxyapatite-binding molecule.

The glycan will be described below.

### Glycan

In the present invention, the term “glycan” refers to a compound in which two or more monosaccharides bind together. In the glycan, two or more monosaccharides bind through glycosidic bonding by dehydration condensation. The glycan is not limited to a compound in which only monosaccharides are linked, and may be a compound in which derivatives of monosaccharides are linked. Alternatively, a monosaccharide and a monosaccharide derivative may be mixed and linked. The glycan may be a linear-chain type in which monosaccharides and monosaccharide derivatives are linearly chained or may be a branched-chain type having a branched chain.

Specific examples of the glycan include glucose, galactose, mannose, fucose, xylose, N-acetylglucosamine, N-acetylgalactosamine, and derivatives thereof. However, there is no limitation to a particular configuration. The glycan may be a degradation product of a polysaccharide, a glycoprotein, a proteoglycan, a glycosaminoglycan, or a glycolipid.

The derivative of the monosaccharide is not particularly limited. For example, the derivative of the monosaccharide may have a structure in which the sugar forming a glycan includes a carboxyl group, an amino group, a sulfate group or a phosphate group, or the sugar forming a glycan is deoxidized. Alternatively, the derivative of the monosaccharide may have a structure in which the amino group, the sulfate group or the phosphate group is substituted with a salt.

Specific examples of the sugar having a carboxyl group include, for example, sialic acid, aldonic acid such as D-gluconic acid, and uronic acid such as iduronic acid and glucuronic acid (D-glucuronic acid).

Specific examples of the sugar having an amino group include, for example, D-glucosamine and D-galactosamine.

Specific examples of the deoxidized sugar include, for example, 2-deoxy-D-ribose.

Specific examples of the sugar having a sulfate group include, for example, phosphorylated sugar.

The glycans described above may be a single type or a combination of two or more types. Among these, it is preferred that the glycan is glycosaminoglycan.

Glycosaminoglycan is formed of polysaccharides of a linear chain type.

Glycosaminoglycan may be in the form of a salt such as sodium salt.

Specific examples of glycosaminoglycan include hyaluronic acid, chondroitin sulfate, keratan acid, heparan acid, heparin, and low molecular weight heparin. However, there is no limitation to a particular configuration. Among these, it is preferred that the glycan be hyaluronic acid, chondroitin sulfate, heparin, or low molecular weight heparin. In addition, when the glycan is low molecular weight heparin, a hydroxyapatite-binding molecule readily binds to the glycan. Also, a drug such as a growth factor readily binds to the glycan. Thus, the low molecular weight heparin is preferred.

In general, heparin refers to heparin having an average molecular weight in a range of 15000 to 20000, inclusive. The low molecular weight heparin refers to a low molecular weight heparin obtained by depolymerizing heparin or a low molecular weight heparin obtained by chemical synthesis. The average molecular weight of the low molecular weight heparin is in a range of 1000 to 10000, inclusive.

A process for measuring the average molecular weight is not particularly limited. A suitable known measuring process may be used. The average molecular weight can be measured, for example, by gel permeation chromatography using a reference substance having a known molecular weight.

The hydroxyapatite-binding molecule will be described below.

### Hydroxyapatite-Binding Molecule

The hydroxyapatite-binding molecule refers to a residue of a compound having a property of readily binding to hydroxyapatite (hereinafter, also referred to as HA), which is a main component of bone.

The phrase “glycan that binds to a hydroxyapatite-binding molecule” of the present invention refers to a state in which a glycan binds to the residue of a compound having a property of readily binding to HA. The HA-binding molecule that binds to a glycan is referred to as a glycan complex.

The compound having a property of readily binding to HA may include, for example, acidic peptide, bisphosphonate, and estradiol. However, there is no limitation to a particular compound.

Bisphosphonate is a compound similar to pyrophosphoric acid and is known as a compound having a high affinity for HA. Estradiol is a type of estrogen.

Among these, it is preferred that the HA-binding molecule be an acidic peptide.

Specific examples of the acidic peptide include, for example, aspartic acid and glutamic acid.

The acidic peptide is a peptide having a carboxyl group in a side chain. When the HA-binding molecule is an acidic peptide, the carboxyl group of the side chain binds to the phosphate of HA. Thus, the acidic peptide binds to HA. In the same manner, when the HA-binding molecule is other than an acidic peptide, the functional group of the HA-binding molecule binds to the phosphate of HA or the like. Thus, the HA-binding molecule binds to HA.

The aspartic acid and the glutamic acid may be formed of a single aspartic acid and a single glutamic acid or may be formed of multiple aspartic acids and multiple glutamic acids that are bonded in the form of a chain. In other words, the acidic peptide may be formed of multiple structural units that bind to each other.

The acidic peptide may be obtained by binding a single aspartic acid to a single glutamic acid. The acidic peptide is obtained by binding aspartic acid and glutamic acid. At least one of the aspartic acid and the glutamic acid may include multiple acids binding to each other. The acidic peptide may be obtained by binding multiple aspartic acids to multiple glutamic acids. Multiple aspartic acids and multiple glutamic acids may bind at random or may bind for each of multiple structural units.

When multiple aspartic acids or multiple glutamic acids bind to each other, it is preferred that only the aspartic acids bind to each other or only the glutamic acids bind to each other.

The upper limit of the number of structural units binding to each other is preferably 20, and more preferably 10. However, there is no limitation to a particular number. The lower limit of the number of structural units binding to each other is preferably 2, and more preferably 5. However, there is no limitation to a particular number.

The acidic peptides described above may be a single type or a combination of two or more types.

### Method for Producing Glycan Complex

A method for binding an HA-binding molecule to a glycan, that is, a method for producing a glycan complex, is not particularly limited. A suitable known method may be used.

Hereinafter, a method for binding the residue of an aspartic acid as an HA-binding molecule to a glycan will be described.

First, a crosslinking agent having two amino groups binds to a carboxyl group or an aldehyde group of a glycan. Specific examples of the crosslinking agent include 1,11-diamino-3,6,9-trioxaundecane. The carboxyl group or the aldehyde group of the glycan and one of the two amino groups of the crosslinking agent undergo a dehydration condensation reaction to form a peptide bond.

Further, the other amino group of the crosslinking agent binds to a bifunctional reagent in which an N-hydroxysuccinimide active ester and a maleimide group, each having a different reactivity, are introduced to opposite ends of the molecule. Specific examples of the bifunctional reagent include Sulfo-SMCC.

The maleimide group of the bifunctional reagent reacts with a sulfhydryl (SH) group biotin standard reagent that binds to an aspartic acid. The procedure described above produces a glycan complex in which the residue of the aspartic acid binds to the glycan.

The glycan complex according to the present invention may further bind to a drug.

Hereinafter, the drug binding to the glycan complex will be described.

### Drug

The drug binding to the glycan complex is not particularly limited. A suitable known drug for bone-related treatment may be used.

The drug includes, for example, a growth factor. A growth factor is a generic term for endogenous proteins that promote proliferation and differentiation of specific cells.

Specific examples of the growth factor include a bone-forming factor, a fibroblast growth factor, a vascular endothelial growth factor, and a hepatocyte growth factor.

Bone-forming factors (hereinafter also referred to as BMP) are a group of proteins identified as molecules that induce or promote the differentiation of bone tissue and cartilage. BMP is also referred to as a bone morphogenetic protein.

Fibroblast growth factors (hereinafter also referred to as FGF) are a group of proteins identified as molecules involved in angiogenesis, wound healing, and embryonic development. FGF plays an important role in the process of proliferation and differentiation of a wide variety of cells and tissues.

Vascular endothelial growth factors (hereinafter also referred to as VEGF) are a group of proteins identified as molecules involved in vasculogenesis and angiogenesis. VEGF is also referred to as a vascular endothelial cell growth factor, a vascular endothelial mitogen factor, a vascular endothelial cell mitogen factor, or the like.

Hepatocyte growth factors (hereinafter, also referred to as HGF) are a group of proteins identified as molecules related to regeneration of livers and kidneys and protection of nerves.

The drugs described above may be a single type or a combination of two or more types. Commercially available drugs may also be used.

The glycan complex according to the present invention may bind to the drugs described above in advance. The binding of the drug in advance omits the task of binding the drug to the glycan complex when the glycan complex is used as a drug carrier. Thus, the glycan complex is readily used as a drug carrier.

Alternatively, when the glycan complex does not bind to the drug in advance, the glycan complex may be configured to bind to the drug when used as a drug carrier. This configuration allows the glycan complex and the drug to be stored separately. Thus, the quality of each of the glycan complex and the drug may be easily maintain in an acceptable state.

In addition, the glycan complex may be used without binding to the drug. More specifically, the glycan complex and the drug may be configured to be each administered to a predetermined position in the body and then bind to each other. This configuration omits the task of binding the glycan complex and the drug.

Hereinafter, a method for binding a drug to a glycan complex will be described.

### Method for Bonding Drug to Glycan Complex

The method for bonding a drug to a glycan complex is not particularly limited. A suitable known method may be used.

In the method for bonding a drug to a glycan complex, for example, a liquid glycan complex and a liquid drug are prepared. The glycan complex and the drug are weighed and mixed at a predetermined mixing ratio and then allowed to rest for a predetermined length of time so that the glycan complex binds to the drug.

Specifically, when the liquid glycan complex and the liquid drug are mixed, a functional group of a protein of the drug, such as an amino group or a carboxyl group, binds to a functional group of the glycan complex, such as a carboxyl group, an aldehyde group, or a sulfate group.

The resting time is preferably one hour or more, and more preferably two hours or more. However, there is no limitation to a particular time.

The content rate of each of the glycan complex and the drug is not particularly limited. When the sum of the two content rates is considered as 100 mass percent, the lower limit of the content ratio of the glycan complex is preferably 50 mass percent, more preferably 60 mass percent, and further preferably 70 mass percent. The upper limit of the content rate of the glycan complex is preferably 95 mass percent, more preferably 90 mass percent, and further preferably 80 mass percent.

In addition, subsequent to the mixing, stirring may be performed.

Instead of mixing a liquid glycan complex and a liquid drug, a powdery glycan complex and a powdery drug may be mixed. The powdery glycan complex and the powdery drug may be prepared, for example, by lyophilizing a liquid glycan complex and a liquid drug.

Instead of simply mixing a liquid glycan complex and a liquid drug, the liquid glycan complex and the liquid drug may be mixed and then impregnated into an artificial bone formed of HA. The impregnation time is preferably thirty minutes or more, and more preferably one hour or more. However, there is no limitation to a particular impregnation time.

As a result of the impregnation into the artificial bone, the glycan complex may bind to the artificial bone in addition to the binding of the glycan complex to the drug. When the glycan complex binds to the artificial bone, the glycan complex can be administered together with the artificial bone to a predetermined position in the body. When the glycan complex binds to the artificial bone in advance, the concentration of the drug around the bone may be increased in a further preferred manner. The powdery glycan complex and the powdery drug may be dispersed in the artificial bone so that the glycan complex and the drug bond to each other in the artificial bone.

### Other Components

The glycan complex may contain components other than those described above. Examples of other components include a preservative, a thickener, a dispersant, a colorant, a surfactant, and a solvent.

The content rate of other components in the glycan complex is preferably 10 mass percent or less, more preferably 5 mass percent or less, and further preferably 3 mass percent or less. However, there is no limitation to a particular content rate.

### Application Category, Dosage Form, and Usage of Glycan Complex

The application category of the glycan complex is not particularly limited and may be used as, for example, a pharmaceutical product, a designated quasi-drug, or a quasi-drug.

The dosage form of the glycan complex is not particularly limited. The glycan complex may be appropriately prepared in a powder form, a solid form, a semi-solid form, a liquid form.

The usage of the glycan complex is not particularly limited. The glycan complex may be applied to a known usage. The usage of the glycan complex include, for example, a drug carrier used in a drug delivery system.

### Operation and Advantages

The operation of the glycan complex of the present embodiment will now be described.

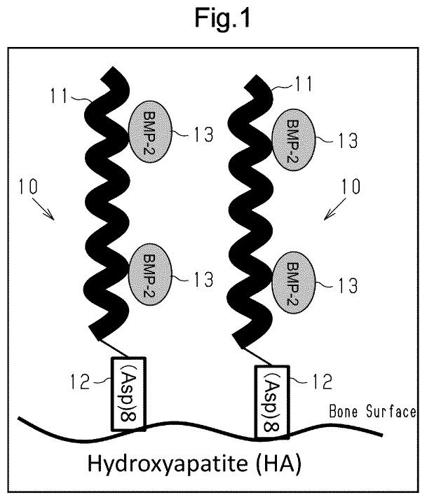

FIG. 1 shows a glycan complex 10 in which the residue of eight aspartic acids (indicated as (Asp)8 in FIG. 1), which corresponds to a HA-binding molecule 12, binds to a glycan 11. In addition, in the glycan complex 10, a bone-forming factor 13 (indicated as BMP-2 in FIG. 1), which corresponds to a drug, binds to the glycan 11. When the glycan complex 10 is administered into the body, the HA-binding molecule 12 binds to a bone, so that the glycan complex 10 is likely to remain on the surface of the bone. Thus, the concentration of the glycan around the bone is increased in a preferred manner.

In addition, the bone-forming factor 13 binds to the glycan 11. Thus, the bone-forming factor 13 is locally present on the surface of the bone. Decreases in the concentration of the drug as time elapses are limited effectively as compared to a prior art in which the concentration of the drug is locally increased due to the low fluidity of the gel. Thus, the concentration of the bone-forming factor 13 around the bone is increased in a preferred manner.

The phrase “around the bone” may refer to, for example, a range within 10 mm from the surface of the bone. However, there is no particular limitation.

The advantage of the glycan complex according to the present embodiment will now be described.

(1) The glycan complex includes a glycan that binds to an HA-binding molecule.
The HA-binding molecule includes at least one of acidic peptide and estradiol.
When the HA-binding molecule binds to a bone, the glycan complex is likely to remain on the surface of the bone. Thus, the concentration of the glycan around the bone is increased in a preferred manner.

(2) The acidic peptide includes at least one selected from aspartic acid and glutamic acid. When the acidic peptide includes at least one selected from aspartic acid and glutamic acid, the concentration of the glycan around the bone is increased in a further preferred manner.

(3) The glycan includes at least one selected from heparin, chondroitin sulfate, and hyaluronic acid. This facilitates binding of the glycan to a drug, such as a growth factor, used for bone-related treatment. Thus, the concentration of the drug around the bone is increased in a preferred manner.

(4) The heparin is a low molecular weight heparin. The low molecular weight heparin is likely to bind to the HA-binding molecule as well as to a drug such as a growth factor. Thus, the concentration of the drug, such as a growth factor, around the bone is increased in a preferred manner.

(5) The glycan binds to at least one selected from the bone forming factor, the fibroblast growth factor, the vascular endothelial growth factor, and the hepatocyte growth factor. Thus, the concentration of the drug around the bone is increased in a preferred manner. When performing bone-related treatment, the effect of the treatment may be enhanced, and the dosage of the drug may be reduced.

(6) The drug carrier includes the glycan complex described above. The drug delivery system uses the glycan complex described above to increase the concentration of a drug at a location around a bone.
Thus, the drug delivery system and the drug carrier used in the drug delivery system increase the concentration of the drug at a local position such as around a bone.

### EXAMPLES

Hereafter, examples will be described in order to specify the configuration and effect according to the present invention. The present disclosure is not limited to the examples described below.

### Test Category 1 (Preparation of Glycan Complex)

### Example 1

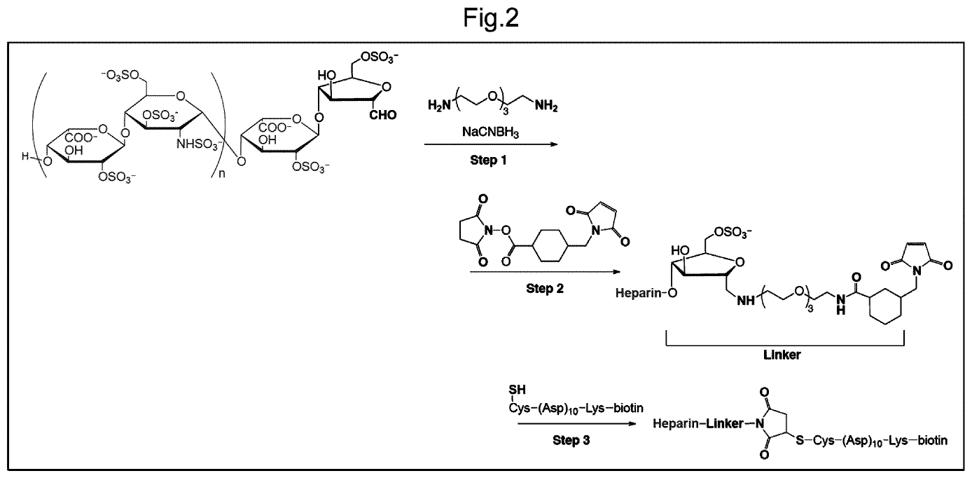

A glycan complex was prepared in accordance with the reaction equation shown in FIG. 2.

Commercially available low molecular weight heparin was used as the glycan. The average molecular weight of the low molecular weight heparin was 5000. Commercially available aspartic acid was used as the HA-binding molecule. In the aspartic acid, eight aspartic acids bind to each other.

As shown in FIG. 2, in step 1, the low molecular weight heparin was dissolved in 2-morpholinoethanesulfonic acid (also referred to as MES), which is used as a buffer. The obtained solution was mixed with 1,11-diamino-3,6,9-trioxaundecane, which is used as a crosslinking agent, and sodium cyanoborohydride, which is used as a reducing agent for imine. The mixed solution was shaken for eighteen hours at a room temperature, underwent centrifugation, and then was washed three times with phosphate-buffered saline (hereinafter also referred to as PBS). The residual material was diluted with PBS to obtain a diluted solution.

In step 2, Sulfo-SMCC, as a crosslinking agent for a bifunctional reagent, was added to the diluted solution. The mixture solution was maintained at a room temperature for thirty minutes, was washed three times with PBS, and then underwent ultrafiltration. The obtained concentrate was dissolved in PBS to obtain a solution.

As a result of steps 1 and 2, 1,11-diamino-3,6,9-trioxaundecane and Sulfo-SMCC bind to the low molecular weight heparin.

In step 3, the above solution was mixed with a sulfhydryl (SH) group biotin standard reagent binding to an aspartic acid. The mixture was maintained at a room temperature for thirty minutes and underwent ultrafiltration. Then, the mixture was washed three times with an acetate buffer. As a result of steps 1 to 3, a liquid glycan complex that bonds to the residue of an aspartic acid was obtained.

### Comparative Example 1

The glycan complex was not used, and only commercially available PBS was used.

### Comparative Example 2

The glycan complex was not used, and only commercially available bone-forming protein (hereinafter, also referred to as BMP-2) was used. BMP-2 is a type of BMP described above and refers to a dimer of BMP.

### Test Category 2 (Evaluation)

When the glycan complex of Example 1 was administered into the body, and the concentration of the glycan around the bone was evaluated. In addition, when the glycan complex of Example 1 binding to the drug was administered into the body, the concentration of the drug around the bone was evaluated.

### Concentration of Glycan Around Bone

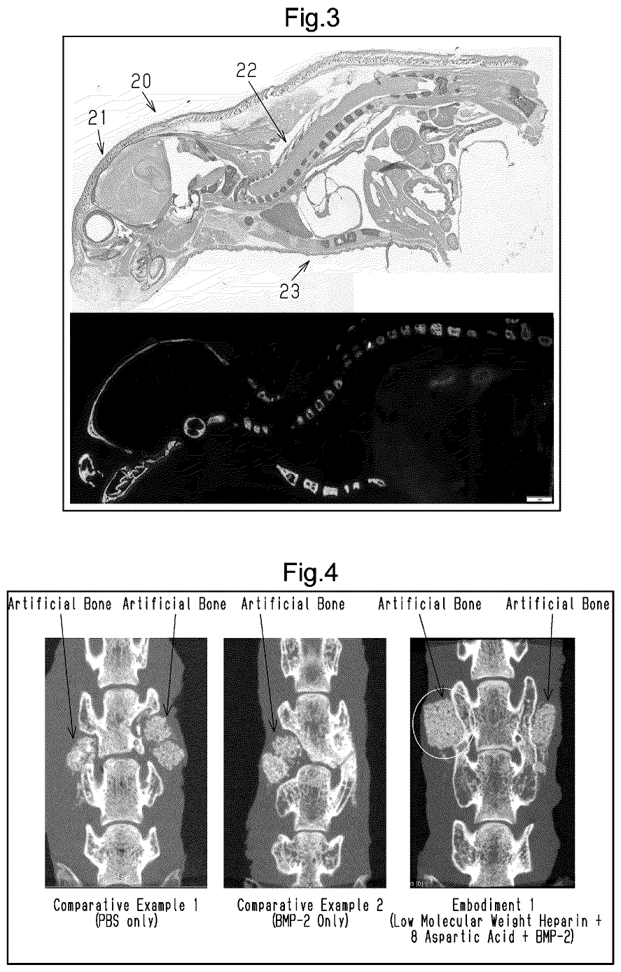

A fluorescent labeling reagent was bound to the glycan complex of Example 1 through a known process. Fluorescein isothiocyanate (also referred to as FITC) was used as the fluorescent labeling reagent. The fluorescent labeling reagent was bound to the glycan complex and subcutaneously injected into a rat. After twenty-four hours, a micrograph of the rat was taken. In the same manner, a fluorescence image of the rat was taken. FIG. 3 shows the results. The process for administering the glycan complex is not limited to subcutaneous injection (subcutaneous administration). A suitable known process used in a drug delivery system may be applied. In the same manner, the glycan complex that binds to the drug may be administered by a known process used in a drug delivery system.

### Concentration of Drug around Bone

The glycan complex of Example 1 (2 nmoL) and BMP-2 (2 μg) were weighed and mixed in a petri dish. After resting for 2 hours, a commercially available artificial bone having a cubic shape with a length, width, and height of about 4 mm was disposed in the petri dish so that the artificial bone was impregnated with the glycan complex and BMP-2. The impregnation was performed for one hour.

The back muscle of an 8-week-old Sprague-Dawley rat (hereinafter also referred to as SD rat) was dissected to expose the transverse processes of the L4 and L5 vertebrae. The transverse processes of the L4 and L5 vertebrae were decorticated using an electric burr. The artificial bone impregnated with the glycan complex and BMP-2 was placed around the decorticated transverse processes. After eight weeks, the L4 vertebrae and the periphery of the L5 vertebrae of the SD rat were observed with a microscope.

In Comparative Example 1, as a control, PBS was only used instead of the glycan complex (2 nmoL) and BMP-2 (2 μg) of Example 1. The other conditions were the same as Example 1 and the test was conducted.

In Comparative Example 2, only BMP-2 (2 μg) was used instead of the glycan complex (2 nmoL) and BMP-2 (2 μg) of Example 1. The other conditions were the same as Example 1 and the test was conducted. FIG. 4 shows the results.

### Evaluation Results

The upper part of FIG. 3 is a micrograph of a rat 20 having hematoxylin and eosin (HE) stain. A skull 21 is located on the left side, and a spine 22 is located on the right side. A sternum 23 is located below the spine 22.

The lower part of FIG. 3 is a fluorescence image taken at the same position as the upper part. In the lower part of FIG. 3, a light color portion indicates a strong fluorescent reaction. The portion indicating a strong fluorescence reaction indicates the presence of a large amount of the fluorescent labeling reagent, that is, the presence of a large amount of the glycan binding to the fluorescent labeling reagent.

From the fluorescence image of FIG. 3, in Example 1, the fluorescence reaction was strong around the bones of the rat such as the skull 21, the spine 22, and the sternum 23. This confirms that the concentration of the glycan around the bone was increased in a preferred manner. That is, the glycan was specifically transported to the bone (HA) as a result of subcutaneous administration.

As shown in FIG. 4, in Comparative Examples 1 and 2, the transverse processes of the L4 and L5 vertebrae remained decorticated. That is, the transverse processes partially remained deficient. In addition, a portion of the artificial bone remained at a position away from the L4 and L5 vertebrae. In Comparative Examples 1 and 2, formation of a bone was not observed between the transverse process and the artificial bone. This confirms that bone fusion was insufficient.

In Example 1, the decorticated transverse processes of the L4 and L5 vertebrae were restored to some degree. More specifically, the artificial bone was attached to the periphery of the transverse processes, and the amount of the artificial bone that was fused is increased as compared to Comparative Examples 1 and 2. This confirms that the bone fusion progressed in Example 1. There is also an indication that the concentration of BMP-2 around the bone was increased in a preferred manner.

### REFERENCE SIGNS LIST

10) glycan complex; 11) glycan; 12) HA-binding molecule; 13) bone-forming factor; 20) rat; 21) skull, 22) spine; 23) sternum

1. A glycan complex, comprising:
a glycan that binds to a hydroxyapatite-binding molecule, wherein the hydroxyapatite-binding molecule includes at least one of acidic peptide and estradiol.

2. The glycan complex according to claim 1, wherein the acidic peptide includes at least one selected from aspartic acid and glutamic acid.

3. The glycan complex according to claim 1, wherein the glycan includes at least one selected from heparin, chondroitin sulfate, and hyaluronic acid.

4. The glycan complex according to claim 3, wherein the heparin is a low molecular weight heparin.

5. The glycan complex according to claim 1, wherein the glycan further binds to at least one selected from a bone forming factor, a fibroblast growth factor, a vascular endothelial growth factor, and a hepatocyte growth factor.

6. A drug carrier comprising: the glycan complex according to claim 1.

7. A drug delivery system that uses the glycan complex according to claim 1 to increase a concentration of a drug around a bone.
